(19) United States
(12) Patent Application Publication
(10) Pub. No.: US 2026/0114808 A1
(43) Pub. Date: Apr. 30, 2026

(54) LOWER LIMBS WEARABLE DEVICE

(71) Applicant: TAIWAN TEXTILE RESEARCH INSTITUTE, NEW TAIPEI CITY (TW)

(72) Inventors: Chien-Fa TANG, NEW TAIPEI CITY (TW); Yu-Chieh LIN, NEW TAIPEI CITY (TW); Wei-An CHEN, NEW TAIPEI CITY (TW); Chien-Lung SHEN, NEW TAIPEI CITY (TW)

(21) Appl. No.: 19/098,025

(22) Filed: Apr. 2, 2025

(30) Foreign Application Priority Data
Oct. 30, 2024 (TW) 113141686

(51) Int. Cl. A61B 5/00; A61B 5/389; D03D 1/00; D03D 13/00; D03D 15/533; D03D 15/56

(52) U.S. Cl. CPC A61B 5/6804; D03D 1/0088; D03D 13/004; D03D 15/533; D03D 15/56; A61B 5/389; A61B 2562/164; D10B 2401/18; D10B 2509/00

(57) ABSTRACT

A lower limbs wearable device includes a flexible textile sleeve. The flexible textile sleeve is weaved by elastic yarns and conductive yarns. The flexible textile sleeve includes knee alignment mark, a lower-front alignment mark, and an upper-front alignment mark. The knee alignment mark is disposed at an outer surface of a middle section of the flexible textile sleeve. The lower-front alignment mark is disposed at an outer surface of a lower section of the flexible textile sleeve. The upper-front alignment mark is disposed at an outer surface of an upper section of the flexible textile sleeve. The flexible textile sleeve further includes a plurality of textile electrodes, textile contacts, and textile wires. The textile wires connect the textile electrodes to the textile contacts, respectively.

### CROSS-REFERENCE TO RELATED APPLICATION

This application claims priority to Taiwan Application Serial Number 113141686, filed Oct. 30, 2024, which is herein incorporated by reference in its entirety.

### BACKGROUND

### Field of Invention

The present disclosure relates to a lower limbs wearable device.

### Description of Related Art

In recent years, man-machine interaction integrates various brain-computer interface technologies such as virtual reality technology, motion sensing, biofeedback technology etc., which develops rapidly in the rehabilitation field. The method of using muscle electric signal feedback to realize man-machine interaction not only increases the participation interest of user on machine interactive interface, but also extracts useful information in complicated bioelectrical signals for the benefit of neuromuscular tracking and evaluating, and improves rehabilitation efficiency.

Current electromyography (EMG) sensing devices typically rely on wearable electrodes attached to the user's body. However, during physical activities, factors such as sweating or muscle contractions often cause the fabric to slip or stretch, leading to electrode displacement. As a result, the measured EMG signals may become distorted and unusable.

### SUMMARY

An aspect of the disclosure provides a lower limbs wearable device, which includes a flexible textile sleeve woven by a plurality of elastic yarns and a plurality of conductive yarns. The flexible textile sleeve includes a knee alignment mark disposed on an outer surface of a middle section of the flexible textile sleeve, a lower front alignment mark disposed on an outer surface of a lower section of the flexible textile sleeve, and an upper front alignment mark disposed on an outer surface of an upper section of the flexible textile sleeve. The knee alignment mark includes a knee cap upper edge position line and a knee cap lower edge position line. The lower front alignment mark includes a tibia position line. A line extended between symmetric axes of the tibia position line and the upper front alignment mark passes through a center of the knee alignment mark. The flexible textile sleeve further includes a plurality of textile electrodes including a first portion of the conductive yarns and disposed on an inner surface of the flexible textile sleeve, a plurality of textile contacts including a second portion of the conductive yarns, and a plurality of textile wires including a third portion of the conductive yarns. The textile contacts are disposed on the inner surface of the flexible textile sleeve, penetrate the flexible textile sleeve, and connect to an outer surface of the flexible textile sleeve. The textile wires connect the textile electrodes to the textile contacts, respectively.

It is to be understood that both the foregoing general description and the following detailed description are by examples, and are intended to provide further explanation of the disclosure as claimed.

### BRIEF DESCRIPTION OF THE DRAWINGS

The accompanying drawings are included to provide a further understanding of the disclosure, and are incorporated in and constitute a part of this specification. The drawings illustrate embodiments of the disclosure and, together with the description, serve to explain the principles of the disclosure.

FIG. 1 is a front view of an outer surface of the lower limbs wearable device according to some embodiments of the disclosure.

FIG. 2 is a back view of the outer surface of the lower limbs wearable device according to some embodiments of the disclosure.

FIG. 3 is a front view of an inner surface of the lower limbs wearable device according to some embodiments of the disclosure.

FIG. 4 is a back view of the inner surface of the lower limbs wearable device according to some embodiments of the disclosure.

FIG. 5 to FIG. 7 are front views of the outer surface of the lower limbs wearable device according to different embodiments of the disclosure, respectively.

FIG. 8 is a partial schematic view of the lower limbs wearable device according to some embodiments of the disclosure.

FIG. 9 to FIG. 12 are fabric weaves of portions of the lower limbs wearable device according to some embodiments of the disclosure, respectively.

FIGS. 13A, 13B, and 13C are graph of average resistance, graph of variability of average resistance, and graph of resistance measured before and after being stretched of the conductive circuit of the lower limbs wearable device, according to some embodiments of the disclosure, respectively.

FIG. 14 is a partial cross-sectional view of the lower limbs wearable device according to some embodiments of the disclosure.

### DESCRIPTION OF THE EMBODIMENTS

Reference will now be made in detail to the present embodiments of the disclosure, examples of which are illustrated in the accompanying drawings. Wherever possible, the same reference numbers are used in the drawings and the description to refer to the same or like parts.

Further, spatially relative terms, such as “on,” “over,” “under,” “between” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

Patients with various medical conditions or those recovering from surgery often require exercise rehabilitation therapy. However, they frequently face challenges in performing rehabilitation at home, such as uncertainty about how to properly conduct exercises or difficulty in verifying whether their movements meet the prescribed guidelines. The disclosed wearable lower-limb device is capable of sensing the wearer's electromyographic (EMG) signals, providing feedback to help determine whether the movements comply with the prescribed standards. Additionally, the disclosed device can also deliver functional electrical stimulation (FES), actively applying electrical currents beneath the skin to stimulate the nervous system, induce muscle contractions, and enhance functional limb movements.

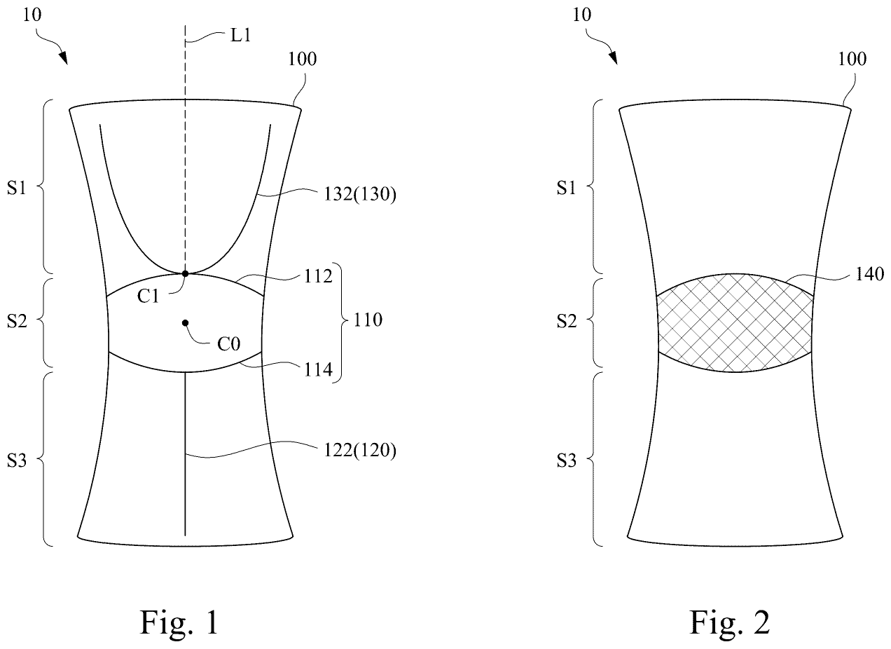

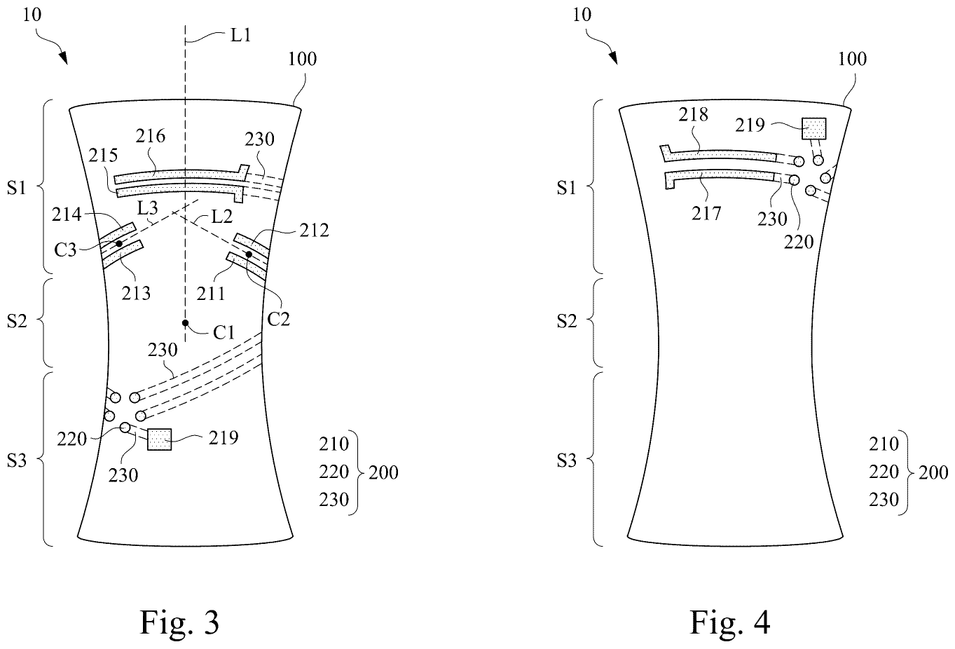

Reference is made to FIG. 1 to FIG. 4, in which FIG. 1 is a front view of an outer surface of the lower limbs wearable device according to some embodiments of the disclosure, FIG. 2 is a back view of the outer surface of the lower limbs wearable device according to some embodiments of the disclosure, FIG. 3 is a front view of an inner surface of the lower limbs wearable device according to some embodiments of the disclosure, and FIG. 4 is a back view of the inner surface of the lower limbs wearable device according to some embodiments of the disclosure.

The lower limbs wearable device 10 includes a flexible textile sleeve 100. The flexible textile sleeve 100 is woven by a plurality of elastic yarns and a plurality of conductive yarns and is a textile that is one-piece formed. The lower limbs wearable device 10 includes a conductive circuit 200, and the conductive circuit 200 is disposed at the inner surface of the flexible textile sleeve 100, in which the inner surface of the flexible textile sleeve 100 that touches wearer's skin when the lower limbs wearable device 10 is worn on the wearer.

The conductive circuit 200 is also woven and is one-piece formed with the flexible textile sleeve 100 such that the electrode pairs of the conductive circuit 200 can be stably secured on the predetermined positions of the desired muscles during movements of the muscles. The accuracy of detecting EMG signals and motion analysis can be improved. Additionally, during wearing the lower limbs wearable device 10, the conductive circuit 200 is stretched along with the flexible textile sleeve 100. Therefore, the conductive circuit 200 is well positioned when the flexible textile sleeve 100 is property worn. The conventional complicated processes of setting each of the electrode pairs can be omitted.

The flexible textile sleeve 100 includes an upper section S1, a middle section S2, and a lower section S3. The upper section S1 corresponds to a thigh portion, the middle section S2 corresponds to a knee portion, and the lower section S3 corresponds to a shank portion. The lower limbs wearable device 10 includes a plurality of alignment marks disposed on the outer surface of the flexible textile sleeve 100, such that the wearer can easily wear the lower limbs wearable device 10 to the correct position. The color of the yarns of the alignment marks is different from the background color of the base fabric for better identifying. The yarns of the alignment marks and the yarns of the base fabric include elastic yarns. The arrangement of the alignment marks is shown in FIG. 1, for example.

The flexible textile sleeve 100 includes a knee alignment mark 110 disposed at the outer surface of the middle section S2. The knee alignment mark 110 includes a knee cap upper edge position line 112 and a knee cap lower edge position line 114. In some embodiments, the knee cap upper edge position line 112 and the knee cap lower edge position line 114 respectively are curved lines, the top of the knee cap upper edge position line 112 points to the upper section S1, and the bottom of the knee cap lower edge position line 114 points to the lower section S3. In some embodiments, widths of the knee cap upper edge position line 112 and the knee cap lower edge position line 114 are wider than the width of the knee cap. For example, the widths of the knee cap upper edge position line 112 and the knee cap lower edge position line 114 laterally cross the middle section S2 of the flexible textile sleeve 100 to make sure that knee caps of wearers of various body shapes can be concluded in the area defined by the knee cap upper edge position line 112 and the knee cap lower edge position line 114.

The flexible textile sleeve 100 includes a lower front alignment mark 120 disposed at the outer surface of the lower section S3. The lower front alignment mark 120 includes a tibia position line 122. The tibia position line 122 is substantially a straight line because the tibia is a straight bone.

The flexible textile sleeve 100 includes an upper front alignment mark 130 disposed at the outer surface of the upper section S1. Unlike the knee has the knee cap and the shank has the tibia, the thigh does not have an observable and obvious bone as an alignment reference. Therefore, the upper front alignment mark 130 has a wider than larger covering range, and the line extending between the tibia position line 122 and the symmetric axis L1 of the upper front alignment mark 130 passes the center C0 of the knee alignment mark 110. In some embodiments, the upper front alignment mark 130 includes an arc position line 132, and a bottom of the arc position line 132 points toward the middle point C1 of the knee cap upper edge position line 112.

By the guidance of the alignment marks such as the knee alignment mark 110, the lower front alignment mark 120, and the upper front alignment mark 130, the wearer can intuitively wear the lower limbs wearable device 10 to the predetermined position, including aligning the knee cap upper edge position line 112 and the knee cap lower edge position line 114 with the knee cap, aligning the tibia position line 122 with the tibia, and pointing the bottom of the arc position line 132 toward the top edge of the knee cap.

In some embodiments, the lower limbs wearable device 10 includes a mesh structure 140 disposed at the back side of the middle section S2 of the flexible textile sleeve 100. The mesh structure 140 is positioned opposite to the knee alignment mark 110. The mesh structure 140 is positioned corresponding to the popliteal. The mesh structure 140 is utilized to increase the permeability and bending flexibility at knee joint when the wearer wears the lower limbs wearable device 10 doing exercise.

As mentioned above, the conductive circuit 200 is also stretched along with the flexible textile sleeve 100, so the conductive circuit 200 is also positioned when the flexible textile sleeve 100 is worn correctly. An example of the arrangement of the conductive circuit 200 is shown in FIG. 3 and FIG. 4.

The conductive circuit 200 includes a plurality of textile electrodes 210, a plurality of textile contacts 220, and a plurality of textile wires 230. All of the textile electrodes 210, the textile contacts 220, and the textile wires 230 are woven by conductive yarns or a combination of conductive yarns and elastic yarns, and the textile electrodes 210, the textile contacts 220, the textile wires 230, and the flexible textile sleeve are one-piece woven. The textile wires 230 are configured to connect the textile electrodes 210 to the corresponding textile contacts 220. The textile contacts 220 are extended from the inner surface to the outer surface of the flexible textile sleeve 100.

The textile electrodes 210 include an electrode pair of vastus medialis 211, 212. The electrode pair of vastus medialis 211, 212 are disposed on the inner surface of the upper section S1 of the flexible textile sleeve 100. In some embodiments, the center C2 of the electrode pair of vastus medialis 211, 212 is positioned 3 cm to 5 cm above the middle point C1 of the knee cap upper edge position line 112 and is 2 cm to 6 cm inward the middle point C1 of the knee cap upper edge position line 112, preferably is about 4 cm above and 3 cm to 4 cm inward the middle point C1 of the knee cap upper edge position line 112. In some embodiments, an angle between a central axis L2 of the electrode pair of vastus medialis 211, 212 and a symmetric axis L1 of the upper front alignment mark 130 (see FIG. 1) is in a range from 110 degrees to 130 degrees, preferably is about 120 degrees such as 121 degrees. The central axis L2 of the electrode pair of vastus medialis 211, 212 is between the electrode pair of vastus medialis 211, 212 and passes the center C2.

The textile electrodes 210 include an electrode pair of vastus lateralis 213, 214 disposed on the inner surface of the upper section S1 of the flexible textile sleeve 100. In some embodiments, the center C3 of the electrode pair of vastus lateralis 213, 214 is 6 cm to 12 cm above the middle point C1 of the knee cap upper edge position line 112 and is 4 cm to 10 cm outward the middle point C1 of the knee cap upper edge position line 112, preferably is 8 cm to 10 cm above and 6 cm to 8 cm outward the middle point C1 of the knee cap upper edge position line 112. In some embodiments, an angle between a central axis L3 of the electrode pair of vastus lateralis 213, 214 and the symmetric axis L1 of the upper front alignment mark 130 is in a range from 100 degrees to 120 degrees, preferably is about 110 degrees such as 108 degrees. The central axis L3 of the electrode pair of vastus lateralis 213, 214 is between the electrode pair of vastus lateralis 213, 214 and passes the center C3.

The textile electrodes 210 include an electrode pair of quadriceps femoris 215, 216 disposed on the inner surface of the upper section S1 of the flexible textile sleeve 100. The electrode pair of quadriceps femoris 215, 216 is disposed above the electrode pair of vastus medialis 211, 212 and the electrode pair of vastus lateralis 213, 214, and the electrode pair of quadriceps femoris 215, 216 is laterally extended between the electrode pair of vastus medialis 211, 212 and the electrode pair of vastus lateralis 213, 214.

The textile electrodes 210 include an electrode pair of hamstring 217, 218 disposed on the inner surface of the upper section S1 of the flexible textile sleeve 100 and opposite to the electrode pair of quadriceps femoris 215, 216. Namely, the electrode pair of quadriceps femoris 215, 216 is disposed at the front side of the flexible textile sleeve 100, and the electrode pair of hamstring 217, 218 is disposed at the back side of the flexible textile sleeve 100.

The textile electrodes 210 further include a plurality of reference electrodes 219. The reference electrodes 219 are disposed on the positions other than corresponding to muscles of quadriceps femoris or hamstring. The reference electrodes 219 are configured to provide reference level or serve as grounding.

The electrode pair of vastus medialis 211 and the electrode pair of vastus lateralis 213, 214 need to be positioned more precisely, because vastus medialis and vastus lateralis are partially covered by the rectus femoris. The quadriceps femoris is a combination of vastus medialis, vastus lateralis, rectus femoris, and vastus internus, so laterally extending the electrode pair of quadriceps femoris 215, 216 between the vastus medialis and the vastus lateralis is sufficient. Similarly, the hamstring is not covered by other muscles, so laterally extending the electrode pair of hamstring 217, 218 in a wide range is also sufficient.

Moreover, the cover range of the lower limbs wearable device 10 includes knee and portions of thigh and shank. The bone size and ratio of adult individuality difference is not obvious, so that the arrangement of the knee alignment mark 110, the lower front alignment mark 120, the upper front alignment mark 130, and the textile electrodes 210 can fit wearers of different genders, ages, heights, and/or weights.

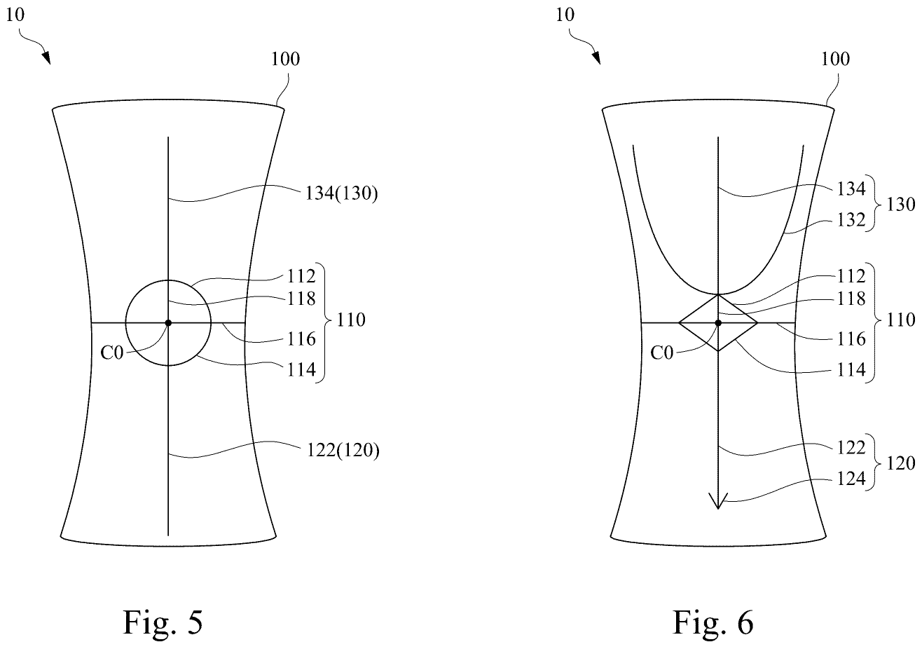

Reference is made to FIG. 5 to FIG. 7, which are front views of the outer surface of the lower limbs wearable device according to different embodiments of the disclosure, respectively. In some embodiments, as shown in the lower limbs wearable device 10 of FIG. 5, the knee cap upper edge position line 112 and the knee cap lower edge position line 114 can be interconnected to construct a circle or an ellipse. The knee alignment mark 110 further includes a horizontal position line 116 and a vertical position line 118 that pass the center C0 of the knee alignment mark 110. The upper front alignment mark 130 includes a straight position line 134, and the straight position line 134 and the tibia position line 122 respectively vertically extended from the vertical position line 118.

In some embodiments, as shown in the lower limbs wearable device 10 of FIG. 6, the knee cap upper edge position line 112 and the knee cap lower edge position line 114 can be interconnected to construct a rhombus. The knee alignment mark 110 further includes the horizontal position line 116 and the vertical position line 118 that pass the center C0 of the knee alignment mark 110, and the horizontal position line 116 and the vertical position line 118 further pass the corners of the rhombus constructed by the knee cap upper edge position line 112 and the knee cap lower edge position line 114. The upper front alignment mark 130 includes the arc position line 132 and the straight position line 134, and the straight position line 134 and the tibia position line 122 respectively vertically extended from the vertical position line 118. Optionally, an arrow 124 is disposed at an end of the tibia position line 122.

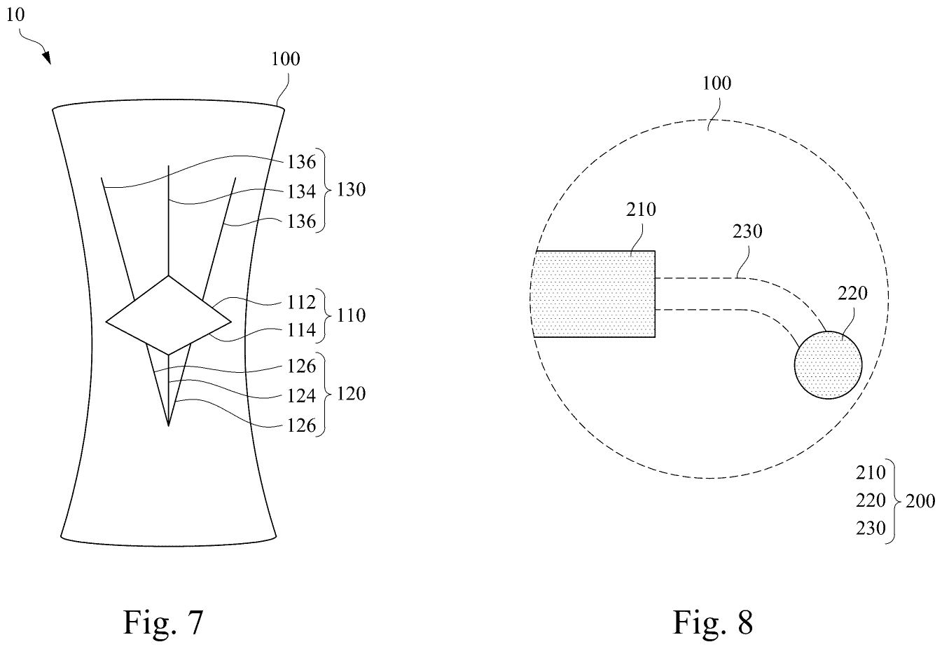

In some embodiments, as shown in the lower limbs wearable device 10 of FIG. 7, the knee cap upper edge position line 112 and the knee cap lower edge position line 114 can be interconnected to construct a rhombus. The upper front alignment mark 130 includes the straight position line 134 and two ascending position lines 136 that are symmetric to the straight position line 134. The extending lines of the ascending position lines 136 pass the tibia position line 122. The lower front alignment mark 120 includes the tibia position line 122 and two descending position lines 126 that are symmetric to the tibia position line 122. The descending position lines 126 and the tibia position line 122 cross at one point.

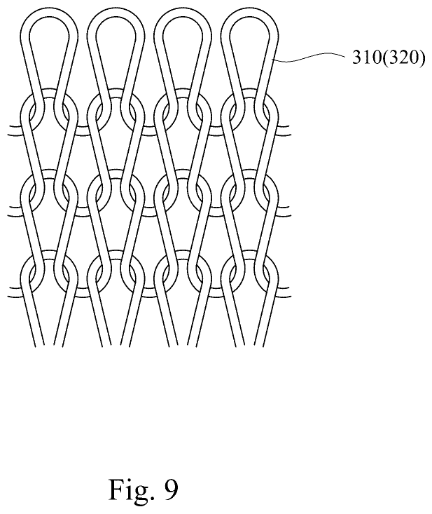

Reference is made to FIG. 8, which is a partial schematic view of the lower limbs wearable device, according to some embodiments of the disclosure. The conductive circuit 200 of the lower limbs wearable device 10 is disposed on the upper section S1, the middle section S2, and the lower section S3 (see FIG. 1) of the flexible textile sleeve 100, so that the fabric weave of the conductive circuit 200 of the lower limbs wearable device 10 is also improved, as discussed below.

The conductive circuit 200 includes textile electrodes 210, the textile contacts 220, and the textile wires 230. The textile electrodes 210 are configured to touch the wearer's skin, the textile contacts 220 are configured to couple to a controller, and the textile wires 230 are configured to connect the textile electrodes 210 to the textile contacts 220, respectively. The flexible textile sleeve 100 for example can be a double-layer fabric structure or a fabric structure that is seamed by two single-layer fabrics, such that the flexible textile sleeve 100 can present different patterns at the inner and outer surfaces.

For example, the base fabric of the flexible textile sleeve 100 (e.g. the fabric without conductive circuit 200) is plain weave constructed all by elastic yarns 310, and the fabric weave thereof is shown in FIG. 9. The elastic yarns 310 may have more than one color so that the alignment marks can be present at the outer surface of the flexible textile sleeve 100 by weaving.

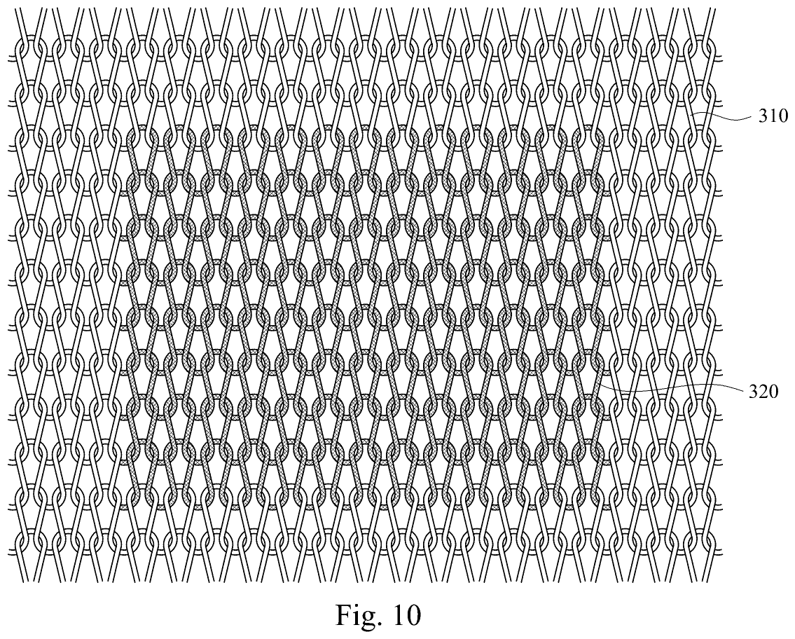

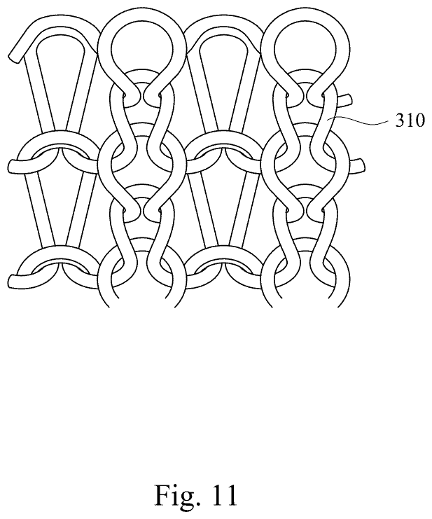

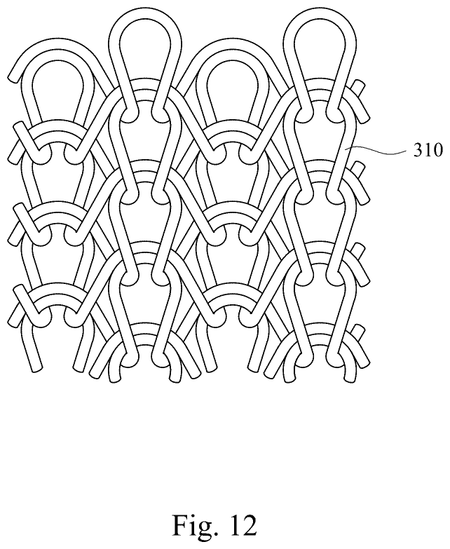

For example, the inner layer of the flexible textile sleeve 100 is intarsia weave, at the textile electrodes 210 and textile contacts 220, and the fabric weave thereof is shown in FIG. 10. The loops of the conductive yarns 320 form the pattern of the textile electrodes 210 and textile contacts 220, and the loops of the elastic yarns 310 form the back ground.

For example, at the textile wires 230 of the inner layer of the flexible textile sleeve 100 is plain weave, and the fabric weave thereof is shown in FIG. 9. At the textile wires 230 of the outer layer of the flexible textile sleeve 100 is cardigan weave formed by the elastic yarns 310 (the fabric weave thereof is shown in FIG. 11) or half cardigan weave formed by the conductive yarns 320 (the fabric weave thereof is shown in FIG. 12). By modifying fabric weave at different positions at the inner and outer surface, the distance between the conductive yarns 320 and skin can be varied. Namely, the conductive yarns 320 of the textile electrodes 210 are more protruded towards the skin side than the conductive yarns 320 of the textile wires 230, and the conductive yarns 320 of the textile wires 230 trend to be hidden in the flexible textile sleeve 100.

Accordingly, in some embodiments, the elastic yarns 310 and a first portion of the conductive yarns 320 are woven by intarsia weave to form the textile electrodes 210, the elastic yarns 310 and a second portion of the conductive yarns 320 are woven by intarsia weave to form the textile contacts 220, and the elastic yarns 310 are weaved by cardigan weave or the third portion of the conductive yarns 320 are weaved by half cardigan weave at the outer layer of the flexible textile sleeve 100 with the third portion of the conductive yarns 320 are weaved by plain weave at the inner layer of the flexible textile sleeve 100 to form the textile wires 230.

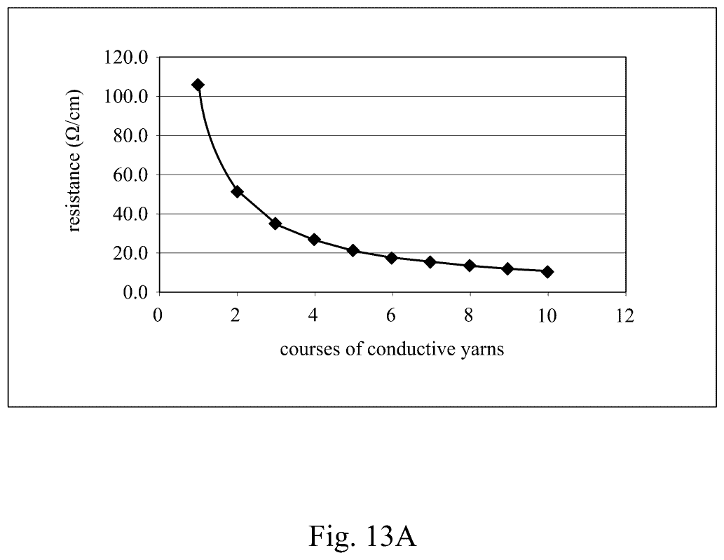

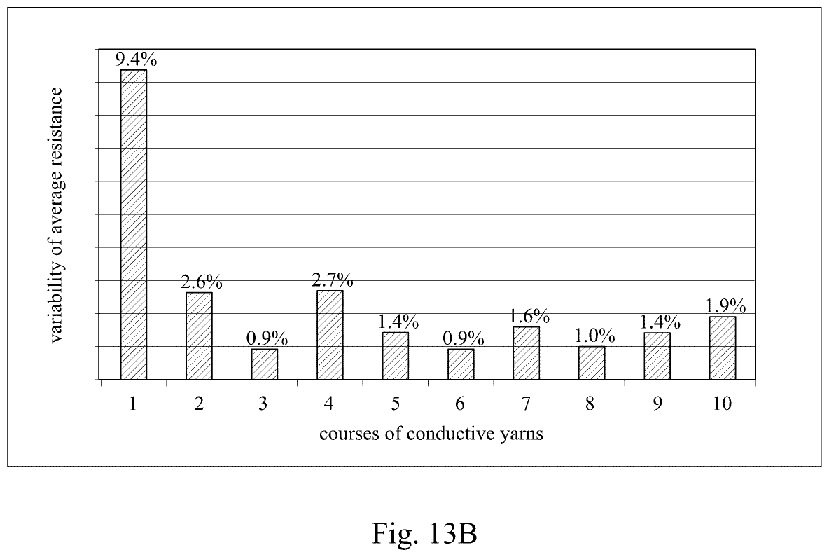

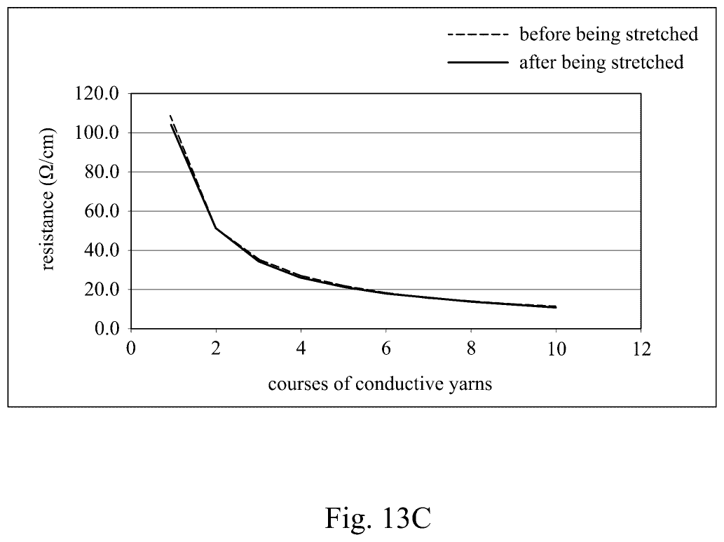

Reference is made to FIGS. 13A, 13B, and 13C, which are graph of average resistance, graph of variability of average resistance, and graph of resistance measured before and after being stretched of the conductive circuit of the lower limbs wearable device, according to some embodiments of the disclosure, respectively, in which FIGS. 13A, 13B, and 13C show average values of samples of different weaving courses.

As shown in FIG. 13A, the average resistance is greatly reduced when the weaving courses is greater than or equal to two. For example, the average resistance is less than 60 Ω/cm when the weaving courses is greater than or equal to two. As shown in FIG. 13B, the variability of average resistance of the conductive circuit is less than 3% when the weaving courses is greater than or equal to two. That means the conductive circuit can provide stable conductive quality. More particularly, the variability of average resistance of the conductive circuit is less than 2% when the weaving courses is greater than or equal to five. As shown in FIG. 13C, the resistance of the conductive circuit before and after being stretched is measured by ASTM D2594 testing method, in which the difference of the resistance of the conductive circuit before and after being stretched is very small (about 1.9% in average). Therefore, the resistance variation before and after the lower limbs wearable device is worn is very small and can be omitted.

According to the testing result of FIGS. 13A, 13B, and 13C, the minimum courses of the conductive yarns of the conductive circuit of the lower limbs wearable device is equal to or greater than two, preferably is three, four, or five, to provide stable conductive quality.

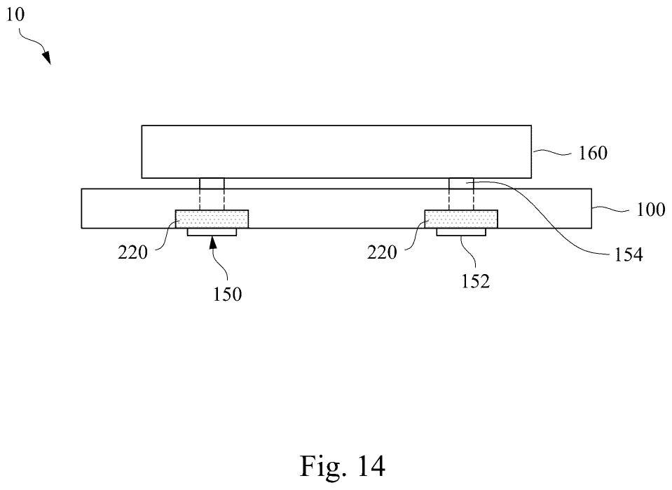

Reference is made FIG. 14, which is a partial cross-sectional view of the lower limbs wearable device according to some embodiments of the disclosure. The lower limbs wearable device 10 includes a plurality of metal connecting pieces 150 disposed on the flexible textile sleeve 100. More particularly, the metal connecting pieces 150 penetrate the flexible textile sleeve 100, the first ends 152 of the metal connecting pieces 150 are connected to the textile contacts 220 respectively, and the second ends 154 of the metal connecting pieces 150 are exposed from the outer surface of the flexible textile sleeve 100. The lower limbs wearable device 10 further includes a control unit 160, and the control unit 160 is coupled to the second ends 154 of the metal connecting pieces 150. The control unit 160 is configured to send electrical pulse signals to the textile electrodes 210 (see FIG. 8) and to receive the EMG signals detected by the textile electrodes 210.

In some embodiments, the control unit 160 includes a wireless transmission module such that the control unit 160 can communicate with other processing unit through the wireless transmission module. In some embodiments, the control unit 160 includes a pulse generator to generate electrical pulse signals to the textile electrodes 210 for electric stimulation in response to the received commands. In some embodiments, the control unit 160 includes an inertial sensor to provide motion path of the wearer to other processing unit for data analysis.

In some embodiments, the metal connecting pieces 150 and the casing of the control unit 160 are coupled by detachable coupling such as buckling or the like. Thus the control unit 160 can be detachably removed from the flexible textile sleeve when the lower limbs wearable device 10 is water washed, and the control unit 160 can survive from water wash.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims and their equivalents.

What is claimed is:

1. A lower limbs wearable device comprising:
a flexible textile sleeve woven by a plurality of elastic yarns and a plurality of conductive yarns, the flexible textile sleeve comprising:
a knee alignment mark disposed on an outer surface of a middle section of the flexible textile sleeve, the knee alignment mark comprising a knee cap upper edge position line and a knee cap lower edge position line;
a lower front alignment mark disposed on an outer surface of a lower section of the flexible textile sleeve, the lower front alignment mark comprising a tibia position line;
an upper front alignment mark disposed on an outer surface of an upper section of the flexible textile sleeve, wherein a line extended between symmetric axes of the tibia position line and the upper front alignment mark passes through a center of the knee alignment mark;
a plurality of textile electrodes comprising a first portion of the conductive yarns and disposed on an inner surface of the flexible textile sleeve;
a plurality of textile contacts comprising a second portion of the conductive yarns, wherein the textile contacts are disposed on the inner surface of the flexible textile sleeve, penetrate the flexible textile sleeve, and connect to an outer surface of the flexible textile sleeve; and
a plurality of textile wires comprising a third portion of the conductive yarns and connecting the textile electrodes to the textile contacts, respectively.

2. The lower limbs wearable device of claim 1, wherein the upper front alignment mark comprises an arc position line, and a bottom of the arc position line points to a middle point of the knee cap upper edge position line.

3. The lower limbs wearable device of claim 1, wherein:
the upper front alignment mark comprises a straight position line and two ascending position lines that are symmetric to the straight position line, and extending lines of the ascending position lines pass through the tibia position line; and
the lower front alignment mark comprises two descending position lines that are symmetric to the straight position line, and the descending position lines cross on the tibia position line.

4. The lower limbs wearable device of claim 1, wherein the knee alignment mark comprises a horizontal position line and a vertical position line that cross at a center of the knee alignment mark.

5. The lower limbs wearable device of claim 1, wherein the textile electrodes comprise an electrode pair of vastus medialis disposed on an inner surface of the upper section of the flexible textile sleeve, wherein a center of the electrode pair of vastus medialis is 3 cm to 5 cm above a middle point of the knee cap upper edge position line and is 2 cm to 6 cm inward the middle point of the knee cap upper edge position line.

6. The lower limbs wearable device of claim 5, wherein an angle between a central axis of the electrode pair of vastus medialis and a symmetric axis of the upper front alignment mark is in a range from 110 degrees to 130 degrees.

7. The lower limbs wearable device of claim 5, wherein the textile electrodes comprise an electrode pair of vastus lateralis disposed on the inner surface of the upper section of the flexible textile sleeve, wherein a center of the electrode pair of vastus lateralis is 6 cm to 12 cm above the middle point of the knee cap upper edge position line and is 4 cm to 10 cm outward the middle point of the knee cap upper edge position line.

8. The lower limbs wearable device of claim 7, wherein an angle between a central axis of the electrode pair of vastus lateralis and the symmetric axis of the upper front alignment mark is in a range from 100 degrees to 120 degrees.

9. The lower limbs wearable device of claim 7, wherein the textile electrodes comprise an electrode pair of quadriceps femoris disposed on the inner surface of the upper section of the flexible textile sleeve, wherein the electrode pair of quadriceps femoris is disposed above the electrode pair of vastus medialis and the electrode pair of vastus lateralis, and the electrode pair of quadriceps femoris is laterally extended between the electrode pair of vastus medialis and the electrode pair of vastus lateralis.

10. The lower limbs wearable device of claim 9, wherein the textile electrodes comprise an electrode pair of hamstring disposed on the inner surface of the upper section of the flexible textile sleeve and opposite to the electrode pair of quadriceps femoris.

11. The lower limbs wearable device of claim 1, further comprising a mesh structure disposed at the middle section of the flexible textile sleeve and opposite to the knee alignment mark.

12. The lower limbs wearable device of claim 1, wherein the textile electrodes are intarsia weave structures woven by the first portion of the conductive yarns and a first portion of the elastic yarns.

13. The lower limbs wearable device of claim 1, wherein the textile contacts are intarsia weave structures woven by the second portion of the conductive yarns and a second portion of the elastic yarns.

14. The lower limbs wearable device of claim 1, wherein an inner layer of the textile wires are plain weave structures woven by the third portion of the conductive yarns.

15. The lower limbs wearable device of claim 1, wherein an outer layer of the textile wires are half cardigan weave structures woven by the third portion of the conductive yarns.

16. The lower limbs wearable device of claim 1, wherein an outer layer of the textile wires are cardigan weave structures woven by a third portion of the elastic yarns.

17. The lower limbs wearable device of claim 1, further comprising a plurality of metal connecting pieces penetrating the flexible textile sleeve, wherein first ends of the metal connecting pieces are connected to the textile contacts, respectively, and second ends of the metal connecting pieces are exposed from the outer surface of the flexible textile sleeve.

18. The lower limbs wearable device of claim 17, further comprising a control unit coupled to the metal connecting pieces, wherein the control unit is configured to send electrical pulse signals to the textile electrodes and to receive signals detected by the textile electrodes.

19. The lower limbs wearable device of claim 1, wherein a minimum courses of the conductive yarns is three.
